(19) United States
(12) Patent Application Publication
(10) Pub. No.: US 2024/0200116 A1
(43) Pub. Date: Jun. 20, 2024

(54) CHACTERIZATION OF BIOLOGICAL ACTIVITY OF FERRITIN PROTEIN BY MASS PRODUCTION USING THE RECOMBINANT VECTOR

(71) Applicants: TURTLEBIO CO., LTD, Yeongcheon (KR); TURTLE BIOTECH, INC., Los Angeles, CA (US)

(72) Inventor: Jang Won CHOI, Daegu (KR)

(21) Appl. No.: 18/066,430

(22) Filed: Dec. 15, 2022

(51) Int. Cl. C12P 21/02; A23J 1/00; A23K 20/147; A23K 50/30; A23K 50/60; A23K 50/75; A61K 9/00; A61K 38/17; C07K 14/47; C12N 15/75

(52) U.S. Cl. CPC C12P 21/02; A23J 1/008; A23K 20/147; A23K 50/30; A23K 50/60; A23K 50/75; A61K 9/0056; A61K 38/1709; C07K 14/47; C12N 15/75; C12N 2800/101; C12N 2820/10

(57) ABSTRACT

Provided is the recombinant vector (pRBASFer-ori) for mass production of ferritin protein and a mass production method of ferritin protein using the same, more specifically, a recombinant vector (pRBASFer-ori) for mass production of ferritin protein, which is a Periserrula leucophryna-derived iron protein; a Bacillus subtilis transformant to which the recombinant vector (pRBASFer-ori) is introduced; and a mass production method using the same. The recombinant vector for mass production of ferritin protein and the mass production method of ferritin protein using the same according to the present invention, has an effect of more efficiently secretion by expressing a ferritin gene obtained from Periserrula leucophryna, and the ferritin-containing culture broth has an excellent biological activity, especially an outstanding biological activities in laying hens, broilers, and weaning pigs, respectively.

### BACKGROUND OF THE INVENTION

### Incorporation of Sequence Listing

This application contains a sequence listing submitted in Computer Readable Form (CRF). The CRF file containing the sequence listing entitled “PK4455886_SequenceListing”, which was created on Dec. 13, 2022, and its size is 10,103 bytes in size. The information in the sequence listing is incorporated herein by reference in its entirety.

### Field of the Invention

The present invention relates to a recombinant vector for mass production of ferritin protein and a mass production method of ferritin protein using the same, more specifically, a recombinant vector for mass production of ferritin protein, which is a Periserrula leucophryna-derived iron protein; a Bacillus subtilis transformant to which the recombinant vector is introduced; and a mass production method using the same.

### Background of the Related Art

Iron is a component of ferritin, transferrin, hemoglobin, myoglobin and so on, which have the functions of transporting and storing oxygen, and plays an important role in a living body in maintaining life as an essential component of electron transport system proteins and peroxidase and others, which are involved in the removal of toxic substances. Therefore, reports have shown that ferritin, which is a representative iron-storing protein in cells, is present in all living species, that is, in various living organisms such as animals, plants, and microorganisms, including humans.

Ferritin in humans and vertebrates is composed of two types of subunits that are genetically and functionally different, which are H (heavy/heart) type and L (light/liver) type. Although there are several isoferritins having different composition ratios in different tissues, it was reported that only H (heavy) type exists in invertebrates. In vertebrates, the L type subunit is involved in the formation of a core of the ferritin molecule, and the H type subunit has a peroxidase activity that is necessary for the absorption of Fe²⁺ and thus has the activity for facilitating the absorption of ferritin and the oxidation of Fe2+. In addition, H type-rich ferritin was reported to have a high level of iron transportation function. Inorganic iron formulation has many limitations in use, because it has a low iron absorption efficiency and causes severe side effects such as gastritis and digestive disorders, considering that the main targets of iron deficiency anemia are infants and pregnant women.

An alternative formulation for this is ferritin, which is used in the treatment of iron deficiency anemia and is known to be much more effective with fewer side effects than inorganic formulations. To date, various studies have been conducted to produce ferritin and utilize the same.

In this regard, KR Patent Registration No. 10-1029765 presents a lentiviral vector capable of simultaneously performing MRI analysis and fluorescence image detection, comprising a nucleic acid molecule encoding a ferritin protein and a fluorescent protein.

In addition, KR Patent Registration No. 10-1811050 presents a fusion polypeptide in which an anti-inflammatory polypeptide is fused at the N-terminus and/or the C-terminus of a ferritin monomer fragment formed by removing a part of the fourth loop and the fifth helix of human-derived ferritin monomer; and a use thereof in the prevention and treatment of inflammatory diseases.

Periserrula leucophryna, which is a type of polychaete that lives in the tidal flat area of Ganghwa Island, a Korea's native living organism that is taxonomically known as a one-genus-one-species organism worldwide. Periserrula leucophryna grows to a maximum length of 2 to 2.5 m, and is known to play a significant role in the recycling in the tidal flat area ecosystem.

Periserrula leucophryna is known to contain H-type ferritin protein, and contains an oxyhydroxied polymer at the core formed at the center of the ferritin protein molecule, thus being capable of biologically polymerizing 1,200 hemoglobin molecules per protein molecule and storing 4,500 iron atoms (trivalent iron, ferric). Therefore, the ferritin protein, as an iron-storing protein, is known to be much more effective with fewer side effects than other inorganic formulations.

Bacillus subtilis is a representative gram-positive bacterium, and since the DNA base sequence in the chromosome has been fully investigated, many factors that are necessary for the gene expression and regulation are known. As a GRAS (generally regarded as safe) strain of which safety has already been proven, the gene manipulation is easy because of the short generation time and the absence of codon bias. In the traditional fermentation process, Bacillus subtilis inherently has a highly efficient protein secretion system, and thus has a high possibility of secreting useful foreign proteins in a large amount, and the recovery of extracellularly secreted proteins is easy. Therefore, Bacillus subtilis is often used as a major host cell for the production of heterologous proteins.

On the other hand, in order to apply ferritin having excellent iron absorption rate and minimum side effects to pharmaceutical industry, food industry and feed industry, the inventors of the present invention has already received a patent registration (KR Patent Registration No. 10-1253505) about a novel ferritin derived from Perinereis aibuhitensis and a gene encoding the same. However, the inventors of the present invention have reached the present invention by the efforts for developing a more efficient high-expression and high-secretion system.

In the present invention, in pursuit of more efficient high-secretion by high-expression of ferritin H-type gene obtained from Periserrula leucophryna, which is a living organism in the tidal flat area, an additional replication origin was introduced to an expression secretion vector comprising key regulatory factors, such as promoter, Shine-dalgarno (SD), operator, signal sequence and replication origin, to increase the copy number of the vector in Bacillus subtilis and thereby optimize the expression and secretion. In addition, mass production of the iron protein was performed under optimal fermentation conditions, and the biological activity of the mass-produced iron protein was confirmed.

### PRIOR ARTS

### Patents

(Patent 001) KR Patent Registration No. 10-1029765 (Lentiviral vectors comprising ferritin gene and use thereof)

(Patent 002) KR Patent Registration No. 10-1811050 (Fusion-polypeptide of anti-inflammatory polypeptide and ferritin monomer fragment and pharmaceutical composition comprising the same)

(Patent 003) KR Patent Registration 10-1253505 (Novel ferritin having iron-binding activity and gene encoding the same)

### SUMMARY OF THE INVENTION

The purpose of the present invention to solve the problem described above is to provide a recombinant vector for mass production of ferritin, which is an iron protein derived from Periserrula leucophryna; a transformant formed by introducing the recombinant vector into Bacillus subtilis; and a mass production method using the same.

According to one aspect of the present invention to solve the technical problem, the present invention provides a recombinant vector for mass production of ferritin, wherein the recombinant vector comprising a gene encoding Periserrula leucophryna-derived ferritin protein represented by the amino acid sequence of SEQ ID NO: 1.

The ferritin protein consists of 174 amino acids, including 17 amino acids of a signal sequence (MATSRQTMPRQNYHEEC).

In the present invention, “vector” refers to a means for expressing ferritin protein by introducing a gene encoding a Periserrula leucophryna-derived ferritin protein, comprises all common vectors, including plasmid vector, cosmid vector, bacteriophage vector, adenoviral vector, retroviral vectors and the like, and is preferably a plasmid vector. In the present invention, the pRBASFer vector disclosed in “Choi, J W (2016) Secretion of ferritin protein of Periserrula leucophyryna in Bacillus subtilis and its feed efficiency” was used as a template; E. coli XL1-Blue MRF (F′, proAB, lacIqZAM15, thi, recA, gyrA , relA, supE, Tn10) (Stratagene, La Jolla, CA) was used as a strain for the replication of the plasmid, and pGEM-T Easy vector(Promega, Madison, USA) was used for the cloning of the PCR product.

According to another aspect of the present invention, the present invention provides a transformant formed by introducing the recombinant vector into a host cell.

The host cell may be selected from any one of Bacillus subtilis, Escherichia coli, and yeast, and preferably, Bacillus subtilis may be used as a host cell for the expression and secretion of a foreign protein. More preferably, Bacillus subtilis LKS87 of “Kim, S. I., J. W. Choi, and S. Y. Lee (1997), Effects of pleiotrophic mutations, degUh and spoOA, on the production of foreign proteins using the heterologous secretion system of Bacillus subtilis, Mol. Cells. 7: 158-164” may be used.

“Transformation” into a host cell herein includes any method of introducing a nucleic acid into an organism, cell, tissue or organ, and may be performed as known in the art by selecting an appropriate standard technique according to the host cell. These methods include electroporation, protoplast fusion, calcium phosphate (CaPO₄) precipitation, calcium chloride (CaCl₂) precipitation, agitation with silicon carbide fiber, agrobacterium mediated transformation, PEG, dextran sulfate, lipofectamine, and drying/inhibition mediated transformation and the like.

According to another aspect of the present invention, the present invention provides a mass production method of Periserrula leucophryna-derived ferritin protein using the recombinant vector.

The mass production method of Periserrula leucophryna-derived ferritin protein using the recombinant vector comprises 1) cloning, into a T vector, a DNA fragment obtained by performing PCR after introducing a replication origin into a vector comprising a ferritin gene of Periserrula leucophryna; 2) obtaining a replication origin fragment by digestion of the cloned T vector with a restriction enzyme; and 3) preparing a recombinant secretion vector by introducing the replication origin fragment into a vector comprising a ferritin gene of Periserrula leucophryna; and 4) culturing, in a medium, a Bacillus subtilis transformant into which the recombinant vector is introduced.

In 1) above, a DNA fragment obtained by performing PCR after introducing a replication origin into a vector comprising a ferritin gene of Periserrula leucophryna is cloned into a T vector, wherein a replication origin is introduced by PCR amplification using a forward primer of SEQ ID NO: 5 and a reverse primer of SEQ ID NO: 6 which have an introduced restriction enzyme site and which are specific to an origin of replication in pRBASFer vector.

In 4) above, a Bacillus subtilis transformant into which the recombinant vector is introduced is cultured in a medium, wherein the medium may be one of PY, LB and MSR, and preferably, PY medium may be used. In addition, the medium may further comprise a nitrogen source and a carbon source, and as a nitrogen source, any one of soy peptone, peptone, potassium nitrate, corn steep liquor, defatted soybean meal, soy protein isolate or a combination thereof may be used, and as the carbon source, any one of glucose, glycerol, barley, starch, molasses, or a combination thereof may be used, but not limited thereto. Preferably, soy peptone may be used as a nitrogen source, and barley as a carbon source. In this case, the nitrogen source may be added in an amount of 1 to 4% (w/v) and the carbon source in an amount of 1 to 3% (w/v).

In this case, the culture process may be performed at a culture temperature of 28 to 35° C., a stirring speed of 150 to 250 rpm, and an air injection rate of 0.5 to 2 vvm for 36 to 96 hours. Preferably, the culture process may be performed at a culture temperature of 30° C., a stirring speed of 200 rpm, and an air injection rate of 1 vvm for 12 to 60 hours

In addition, according to another aspect of the present invention, the present invention provides a ferritin protein produced by the mass production method of Periserrula leucophryna-derived ferritin protein.

In addition, according to another aspect of the present invention, the present invention provides a composition to be added to main feed, comprising the Periserrula leucophryna-derived ferritin protein.

The mixing ratio of the ferritin protein in the composition to be added to main feed may be designed in consideration of nutritional state, the number of servings, the amount of serving and so on, and the ferritin protein may be added in an amount of 0.01 to 5 parts by weight based on 100 parts by weight of the feed or drinking water, preferably, 0.01 to 3 parts by weight, and more preferably, 0.05 to 1.5 parts by weight.

In addition, the composition to be added to main feed may be provided in the form of powder, liquid, or suspension, and the formulation may be selected in consideration of the animal's feed and drinking water ingestion characteristics, the environment, and factors that can minimize the loss of ferritin protein during ingestion. As a specific example, liquid type of the feed may be provided after mixing with drinking water or powder type of the feed (freeze dried powder)can be provided to solid type of main feed.

The composition to be added to main feed is used as a feed additive for Korean beef cattle, dairy cows, pigs, laying hens, broilers, and ducks, and preferably, may be used as a feed additive for laying hens.

In addition, the composition to be added to main feed may be applied without limitation in any fields requiring iron supply rather than a feed additive for animals. Specifically, the composition to be added to main feed may be applied to plant fertilizer additives and the ferritin protein of the composition may be processed to functional foods, pharmaceutical products and the like.

As described above, the recombinant vector for mass production of ferritin protein and the mass production method of ferritin protein using the same according to the present invention has an effect of more efficiently secretion by expressing a ferritin gene obtained from Periserrula leucophryna, which is an invertebrate.

In addition, according to the recombinant vector for mass production of ferritin protein and the mass production method of ferritin protein using the same according to the present invention, the ferritin-containing culture broth has an excellent biological activity, especially an outstanding biological activity in laying hens, broilers, and weaning pigs.

In addition, the recombinant vector for mass production of ferritin protein and the mass production method of ferritin protein using the same according to the present invention has an effect of being applicable to not only feed but also food industry and pharmaceutical material industry for iron deficiency prevention and treatment.

### BRIEF DESCRIPTION OF THE DRAWINGS

FIG. 1 illustrates the cDNA sequence of Periserrula leucophryna ferritin and an amino acid sequence derived from the sequence.

FIG. 2 illustrates the construction strategy of pRBASFer-ori vector and the cleavage map of the recombinant vector for mass production of ferritin protein according to the present invention.

FIG. 3(A) and 3(B) illustrates the analytical results from SDS-PAGE and Western blotting showing secretion efficiency of the ferritin protein by the recombinant vectors (A) pRBASFer and (B) pRBASFer-ori in Bacillus subtilis for mass production of ferritin protein according to the present invention.

FIG. 4(A) and 4(B) illustrates the analytical results from SDS-PAGE and Western blotting showing the secretion efficiency of ferritin protein by the change of the (A) nitrogen source and (B) carbon source of the medium for culturing the Bacillus subtilis transformant with the recombinant vectors (pRBASFer-ori) for mass production of ferritin protein according to the present invention.

FIG. 5(A) and 5(B) shows the ferritin production of the Bacillus subtilis transformant containing pRBASFer-ori for mass production of ferritin protein over incubation time with (A) the plot showing the secreted ferritin protein and the cell growth according to the time interval, and (B) the analytical results from SDS-PAGE and Western blotting.

FIG. 6(A) and 6(B) shows the ferritin production of the Bacillus subtilis transformant containing pRBASFer-ori for mass production of ferritin protein over incubation time in the case of mass culture (50 L jar-fermenter) with (A) the plot showing the secreted ferritin protein and the cell growth according to the time interval, and (B) the analytical results from SDS-PAGE and Western blotting.

FIG. 7(A), 7(B) and 7(C) shows the effect of the ferritin protein produced by the mass production method of ferritin protein according to the present invention on the quality of eggs (eggshell color, egg weight, Haugh unit (freshness)), wherein FIG. 7(A) is a plot showing the effect of the ferritin protein on the quality of eggs (eggshell color, egg weight, Haugh unit (freshness)) during 6 weeks; FIG. 7(B) shows the 6-weeks change of the color of the eggshell treated with the ferritin protein; and FIG. 7(C) is a plot showing the iron content in the eggshell and egg yolk measured by using Inductively Coupled Plasma (ICP) and the change over time.

FIG. 8(A) and 8(B) shows photographs representing the color change of (A) the eggshell and (B) the egg yolk, in Week 1 to Week 4, of the eggs produced by feeding the ferritin protein produced by the mass production method of ferritin protein according to the present invention to laying hens.

The patent or application file contains at least one drawing/photograph executed in color. Copies of this patent or patent application with color drawing(s)/photograph(s) will be provided by the Office upon request and payment of the necessary fee.

### DETAILED DESCRIPTION OF THE EMBODIMENTS

Specific features and advantages of the present invention will be described in details hereinafter with reference to the accompanying drawings. Prior to this, when it is determined that a detailed description of a function and a configuration related to the present invention may unnecessarily obscure the gist of the present invention, a detailed description thereof will be omitted.

Hereinafter, the present invention will be described in details hereinafter with reference to Examples. However, the following Examples are intended to specifically illustrate the present invention, and the present invention is not limited thereto.

### 1. Materials and Experimental Methods

### 1.1. Materials and Reagents

Restriction enzymes and T4 DNA ligase were purchased from Elpisbio (Daejeon, Korea), Taq polymerases from Applied Biosystem (Foster, CA, USA), NEB (Beverly, MA, USA) and bacto-agar, and tryptone and yeast extract from Merck (Lutterworth, Germany), and oligonucleotides were synthesized at Bioneer (Daejeon, Korea).

Immobilon-P PVDF membrane was purchased from Millipore (Bradford, MA, USA); pGEM-Teasy cloning kit and Wizard Plus SV Minipreps DNA purification kit from Promega (Madison, Wisconsin USA); QIAEX agarose gel extraction kit from Qiagen (Hilden, Germany); ECL Western blotting system from Amersham Pharmacia Biotech (Piscataway, NJ, USA); SDS, NaCl, RNaseA, ampicillin, and kanamycin from Sigma-Aldrich (St Louis, MO, USA), and polyclonal antibody from Santa Cruz Biotechnology (Santa Cruz, USA). Other reagents used were of analytical grade.

### 1.2. Design of cDNA Library of Periserrula leucophryna

TRIzol reagent (Sigma, USA), QIAGEN RNA extraction kit, and guanidinium/phenol method (Chomczynski and Sacchi, 1987) were used to isolate total RNAs from the tissues of Periserrula leucophryna (tidal flat area in Ganghwa Island). Purification of poly(A⁺)RNA from the extracted total RNAs was performed by using oligo-dT cellulose (Stratagene, USA) chromatography (Collaborative Biomedical Products, USA) according to the method of Sambrook (Sambrook et al., 1989).

The isolated and purified poly(A⁺)RNA was used to prepare a cDNA library, and the cDNA library was prepared by the according to the manufacturer's instructions (Stratagene, USA). The results showed that the efficiency of generating recombinant plaques (white color) was 8×10⁵ pfu/μg. The primary library, which was unstable, was amplified in E. coli XL1-Blue MRF′ host strain, and the measured titer of the amplified cDNA library was 5×10¹⁰ pfu/mL.

The gene expressing the ferritin of Periserrula leucophryna was amplified by PCR from the prepared cDNA library and then cloned, that is, the amplified ferritin gene was inserted to the pGEM T Easy plasmid (Promega, USA), which is replicated in Escherichia coli.

After the PCR, the amplified DNA fragment was fractionated and confirmed by electrophoresis on 1% agarose gel (in TAE buffer solution), and the amplified DNA fragment was separated from the gel, subcloned into the pGEM T easy plasmid. Then, the base sequence was determined, and the amino acid sequence to be translated therefrom was confirmed.

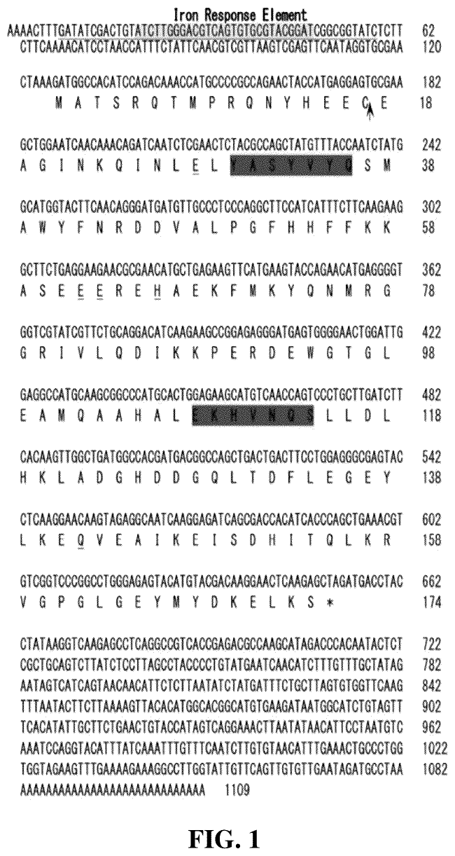

FIG. 1 illustrates the cDNA gene sequence of Periserrula leucophryna ferritin and an amino acid sequence derived from the sequence. The cloned ferritin gene had a base sequence of SEQ ID NO: 2. The entire DNA size was 1,109 bp, and the size of the open reading frame, including the stop codon (TAG), was 525 bp as shown in SEQ ID NO: 3.

The 5′-UTR, which is underlined, has an iron response element (IRE) sequence (−110 to −81, ATCTTGGGACGTCAGTGTGCGTACGGAT) of SEQ ID NO: 4, which forms a stem-loop structure for the binding of an iron regulatory protein (IRP).

The arrow indicates a signal peptide cleavage site, and the amino acid residues used in the synthesis of a degenerated primer are shown as a bold shaded text. The stop codon is asterisked (*) and methylation-related amino acids were underlined. The nucleotide and the amino acid sequence of the ferritin DNA have been registered to the GenBank databases with the registration numbers DQ207752 and ABA55730, respectively.

### 1.3. Bacterial Strains, Plasmid and Medium

E. coli XL1-Blue MRF (F′, proAB, lacIqZΔM15, thi, recA, gyrA, relA, supE, Tn10) (Stratagene, La Jolla, CA) was used as a strain for the proliferation of the plasmid, and pGEM-T Easy Vector (Promega, Madison, USA) was used for the cloning of the PCR product. Bacillus subtilis LKS87 (nprR2, nprE18, aprA3, amyE) strain, which was constructed by a mutation experiment by gene conversion from Bacillus subtilis 168 (wild type, GRAS and genome sequenced strain), was used as a host cell for the expression and secretion of foreign protein.

A pRBASFer-ori expression and secretion vector containing a ferritin gene was prepared in the present invention by further adding a replication origin to the existing pRBASFer (Ampr, Neor, bler, repB, ColEl ori, aprE-P, apr signal sequence, ferritin gene) vector.

An E. coli transformant containing a recombinant vector was cultured in Luria-Bertani (LB) medium at 37° C. at 200 rpm for 16 hours after adding ampicillin (50 μg/mL), and Bacillus subtilis containing the recombinant vector was pre-cultured in LB medium containing kanamycin (50 μg/mL) at 37° C., and then cultured in PY medium containing kanamycin (50 μg/mL) at 30° C. after 1% inoculation.

### 1.4. Construction of Recombinant Secretion Vector and Transformation of Bacillus subtilis

In order to introduce an additional replication origin, a set of oligonucleotide primers of SEQ ID NO: 5 and SEQ ID NO: 6 (Forward primer: 5′GGGACATGTTCTTTCCTGCGTTATCCCCTG3′, Reverse primer: 5′CCCGATATCCTATTTAGAATATTGTTTAGT3′), which were prepared to be specific to the origin sequence, were used to amplify DNA by PCR (Techne, Vantaa, Finland). The replication origin was amplified by PCR under the conditions of 95° C.-5 min, 45° C.-1 min, 72° C.-10 min (1 cycle); 95° C.-1 min, 45° C.-30 sec, 72° C.-1.5 min (29 cycles); and 72° C.-10 min (1 cycle). The PCR product was fractionated in 1% agarose gel to recover a DNA fragment (Qiaquick gel extraction kit, Hilden, Germany), which was then cloned into pGEM-Teasy vector (Promega, Madison, WI). To construct a recombinant vector having an additional replication origin, the pRBASFer was filled after MluI enzyme treatment, and was cleaved with PciI enzyme. The T-pRBori clone containing the gene of the replication origin was cleaved with EcoRV and PciI to obtain a DNA fragment of 508 bp from gel.

The obtained DNA fragment was ligated to the pRBASFer, transferred into E. coli XL-1 Blue MRF′ and right clone was screened. The introduced gene was confirmed by treating with restriction enzymes (EcoRV and PciI), and the finally confirmed clone was named as pRBASFer-ori (7.4 kb). The transformation into Bacillus subtilis was performed by the method of Sadaie and Kada (Sadaie, Y. and T. Kada (1983) Formation of competent Bacillus subtilis cells. J. Bacteriol. 153:813-821) using SPMM-I (Spizizen's minimal medium) and SPMM-II media. The transformant was screened in LB solid medium containing kanamycin (50 μg/mL), and then used for the expression and secretion of ferritin protein.

### 1.5. Protein Expression, Secretion and Mass CUlture

The transformant of Bacillus subtilis LKS87 containing the pRBASFer-ori expression vector to which an additional replication origin was added was cultured in a batch culture (in 500 mL baffled flask) to optimize the ferritin production according to the medium type, temperature and incubation time. Under the optimized conditions obtained from the culture, mass production was performed by using a 5 L jar-fermenter (KoBiotech, Korea) and a 50 L fermenter.

### 1.6. SDS-PAGE and Western Blot Analysis

The obtained culture broth was concentrated directly or by ethanol precipitation, suspended in a 2×sample buffer (0.05 M Tris-HCl, pH 6.8, 0.1 M DTT, 2% SDS, 10% glycerol, and 0.1% bromophenol blue), and heated at 100° C. for 10 minutes. Then, the resulting suspansion was fractionated by 12% SDS-PAGE (sodium-dodecyl sulfate polyacrylamide gel electrophoresis), and quantified by using Bradford protein assay kit (Bio-Rad, Hercules, CA, USA) with BSA (bovine serum albumin) as a standard. After fractionating the proteins in the culture broth by SDS-PAGE, the proteins in SDS-PAGE were transferred to the PVDF membrane by using a Trans-Blot SD apparatus (Bio-Rad, Hercules, CA, USA), and then skim milk was added to TBST (10 mM Tris-HCl, pH 7.5, 150 mM NaCl, 0.1% Tween-20) at a concentration of 5% to perform overnight blocking at 4° C. After that, the primary antibody, which was a rabbit polyclonal anti-ferritin antibody (1:1,000 dilution, Santa Cruz Biotechnology, Dallas, Texas), was treated to a reaction for 1 hour incubation, and then the unbound primary antibody was washed with a TBST solution to remove. Then, the secondary antibody, which was a horseradish peroxidase-conjugated anti-rabbit IgG secondary antibody (1:1000, Santa Cruz Biotechnology), was added to a reaction for 1 hour more incubation, followed by final washing with TBST solution and the membrane was exposed to an X-ray film by using ECL™ Western blotting detection system (Amersham Pharmacia Biotech, USA). The blot was scanned to perform indirect comparative measurement of the protein concentration by using Chemilmager™ (Alpha Innotech Corporation, San Leandro, USA).

### 1.7. Confirmation of Biological Activity by Using Laying Hens

After performing mass production of Bacillus subtilis, transformed with pRBASFer-ori, in an optimized medium by using a 50 L fermenter, the bacteria were removed by using a continuous centrifuge and the fermentation broth was recovered. The expressed and secreted ferritin was fractionated by SDS-PAGE, and identified by Western blotting and finally quantified by the Bradford method (Laemmli, UK (1970) Cleavage of structural proteins during the assembly of the head of bacteriophage T4. Nature. 227: 680-685). The ferritin-containing fermentation broth was diluted to an appropriate concentration (10 μg/mL), and then the diluted broth was added at a concentration of 0.1% to drinking water at a laying hen farm (Daegu University Farm) equipped with an automatic water supply facility, and fed to the laying hens (ISA Brown, 80 weeks of age, 300 laying hens) for 6 weeks. After the feeding, 60 eggs were randomly selected every week, and the eggshell color, egg weight, and Haugh unit (HU) were measured qualitatively and quantitatively with those of the control group for comparative analysis.

### 1.8. Quantification of Iron (Ferric) by High-Frequency Inductively Coupled Plasma Emission Spectroscopy (ICP)

The collected eggshells and egg yolks were entrusted to the Research Center for Instrumental Analysis at Daegu University to measure the iron content by using an inductively coupled plasma (ICP)-OES system device. The pretreatment process was performed by using a microwave (CEM Corporation, Charlotte, USA) in which 0.5 g of eggshell was added to 10 mL of nitric acid and the resulting mixture was heated on a hot plate at 190° C. for 20 minutes. Then, 50 mL of deionized water was added to the pretreated eggshell to analyze the iron content through the high frequency inductively coupled plasma emission spectroscopy (ICP) by using the OPTIMA 7300DV ICP-OES SYSTEM (Carnation, Washington, USA).

### 1.9. Biological Activity of Ferritin in Broilers

To confirm the biological activity of ferritin in broilers (8 to 10 weeks of age), the survival rate, average daily gain, feed efficiency, and production index were checked while feeding and observing for about 1 month.

The biological activity was compared between ferritin supply in a liquid type and a powder type. In the case of a liquid type, a ferritin composition containing 5 mg of ferritin per 1 L of the liquid feed was supplied to drinking water to a final concentration of 0.1%. In the case of a powder type, a ferritin composition containing 5 mg of ferritin per 1 kg of wheat sorts was supplied to a final concentration of 0.1% at the time of feeding the main feed.

### 1.10. Biological Activity of Ferritin in Pig Breeding

The biological activity was confirmed in weanling piglets of three-way crossbreeds (Landrace×Yorkshire×Duroc). The piglets were bred for 6 weeks, while supplying corn-soybean meal as a basal diet, and water on voluntary intake.

The mass-produced ferritin was added as a feed additive to confirm the effect on growth capacity and blood characteristics. The growth capacity was measured by checking the average daily gain, average daily feed intake, and feed efficiency depending on the ferritin concentration. With regard to the blood characteristics, WBC, RBC, lymphocyte and hemoglobin were measured by using an automatic hematology analyzer (ADVID120, Bayer, USA).

### 2. Results and Discussion

### 2.1. Construction of Secretion Vectors Containing Additional Replication Origins

In order to amplify the copy number by additionally introducing the own replication origin of the pRBAS-Fer (6.9 kb) vector having an alkaline protease promoter (0.45 kb), an alkaline protease signal sequence (87 bp), and a ribosome binding sequence (GGAGAGGG), replication origin-specific primers (Ori-F and Ori-R) to which restriction enzyme sites (PciI and EcoRV) were introduced were used to perform PCR with the pRBAS-Fer vector as a template, and 0.5 kb DNA fragments were obtained and cloned to pGEM-Teasy vector for screening. Then, after confirming the introduced replication origin sequence by DNA base sequencing, the resulting vector was named as T-pRBori (3.51 kb).

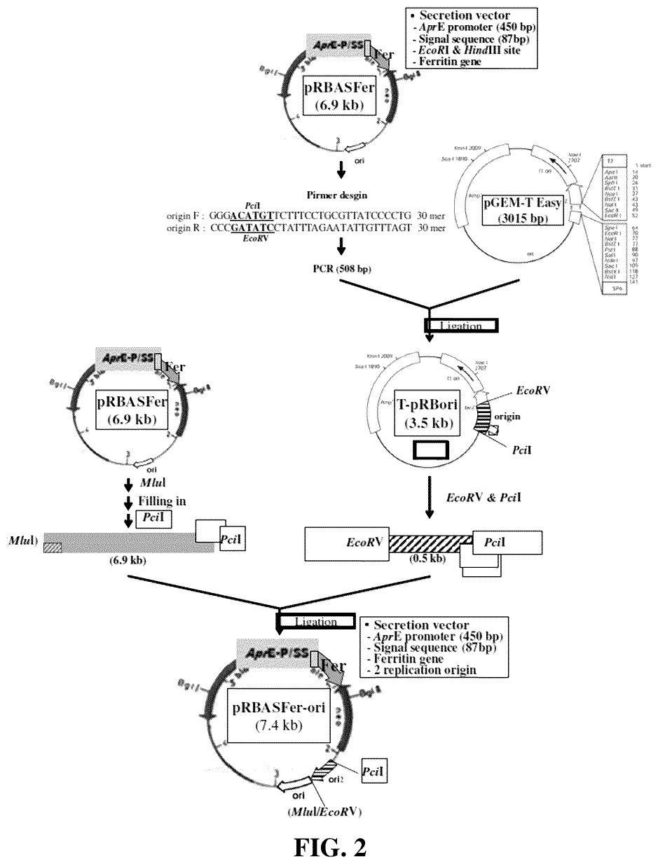

FIG. 2 illustrates the construction strategy and the cleavage map of the recombinant vector for mass production of ferritin protein according to the present invention.

The existing vector pRBAS-Fer was digested with MluI, filled with Klenow enzyme, and digested again with PciI to prepare DNA fragments on agarose gel. In addition, the T-pRBori vector was digested with EcoRV and PciI to prepare a replication origin fragment of 508 bp, which was then ligated with the linearized vector and transformed into E. coli to obtain transformants. A recombinant secretion vector to which replications origins were introduced in a tandem arrangement was constructed by screening of the transformants through DNA size selection and base sequencing, and the vector was named as pRBASFer-ori (7.43 kb). The constructed recombinant secretion vector was amplified in E. coli to be used for the transformation of Bacillus subtilis.

### 2.2. Optimization of Protein Expression and Secretion in Bacillus subtilis

The recombinant secretion vector pRBASFer-ori was introduced into B. subtilis LKS87 by the Sadaie and Kada method using SPMM-I (Spizizen's minimal medium) and SPMM-II media. After screening the transformants in the LB solid medium containing kanamycin (50 μg/mL), the plasmid was isolated to reconfirm the replication origin introduced by the PCR amplification. The ferritin gene was encoded for 157 amino acids of mature form, and predicted for extracellularly secretion of ferritin (18 kDa) protein (159 amino acids) including two amino acids, AAG (Lys) and CTT (Leu), derived from the HindIII restriction enzyme site introduced to the amylase signal sequence ligation site. However, since the addition of the two amino acid could affect the ferritin activity, to remove the coding bases of the two amino acids, specific primer sets were synthesized and the two codons were removed by PCR technique(data not shown). The original mature form of ferritin was secreted after the amylase signal sequence (29 amino acids) was accurately processed and removed by a peptidase.

FIG. 2 shows the secretion efficiency of ferritin protein by the recombinant vectors pRBASFer and pRBASFer-ori in Bacillus subtilis, wherein lane 1: MW marker; lane 2: pRBAS vector; lane 3: ferritin (positive control); and lanes 4-8, transformant 1-5, and the arrow indicates the position of ferritin.

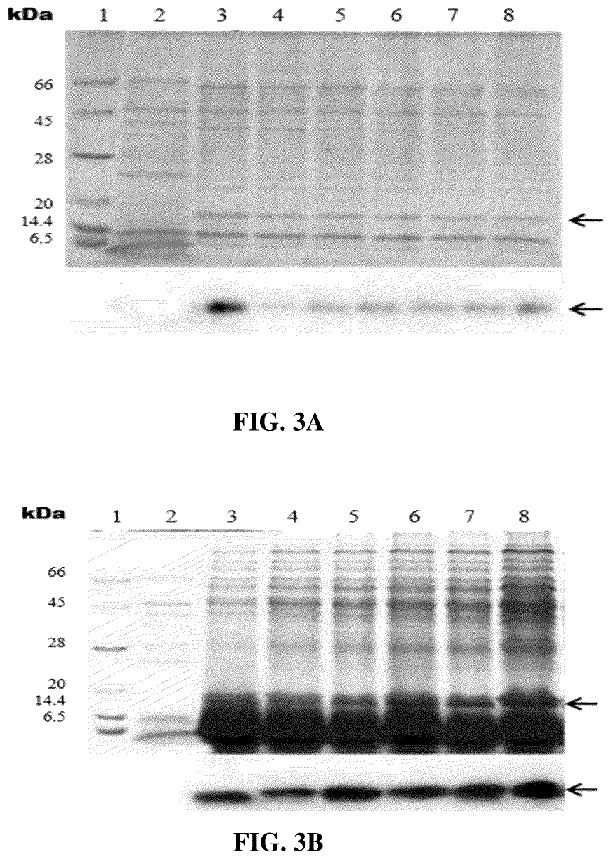

To compare the secretion efficiency of ferritin in transformants (5 types each) obtained by introducing recombinant pRBASFer and pRBASFer-ori (in this study) into B. subtilis LKS87, respectively, the transformants were activated in a 10 mL LB medium containing kanamycin (50 μg/mL), and then the cultured in a 500 mL baffled flask (100 mL working volume) by using a PY liquid medium at 30° C. for 48 hours, and the amount of the secreted ferritin was compared by using a densitometer in SDS-PAGE. In the transformants of Bacillus subtilis containing the existing vector, pRBASFer (No. 1-5), ferritin was secreted in an amount up to 10%-11% of the total proteins, but the amount was increased to 15%-18% of the total proteins in the Bacillus subtilis transformants containing pRBASFer-ori (No. 1-5), indicating that the secretion efficiency of ferritin protein was increased by 1.4 to 1.8 times in average (shown in FIG. 3(A) and 3(B). These results are considered as the effect of introducing an additional replication origin, and the increase of the copy number enhanced the amount of the RNA transcript, thereby increasing the amount of the secreted protein. Among the Bacillus subtilis transformants containing pRBASFer-ori shown in FIG. 3(B), the transformant No. 5 strain, which showed the best secretion efficiency of ferritin protein (up to 18% of the total proteins), was used to investigate the ferritin production by the change of the nitrogen and carbon sources.

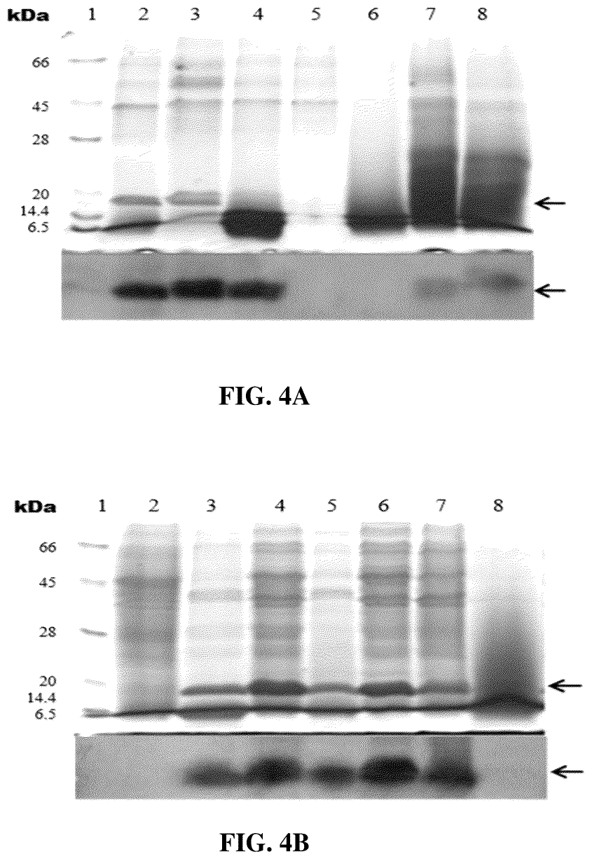

FIG. 4(A) and 4(B) shows secretion efficiency of ferritin protein by the change of the nitrogen and carbon sources of the medium for culturing the Bacillus subtilis transformant, wherein (A) shows the secretion efficiency depending on the nitrogen source and (B) shows the secretion efficiency depending on the carbon source.

In (A), lane 1, MW marker; lane 2, ferritin (positive control); lane 3, soy peptone; lane 4, peptone (Merck); lane 5, potassium nitrate; lane 6, corn steep liquor; lane 7, defatted soybean meal; and lane 8, soy protein; and in (B), lane 1, MW marker; lane 2, pRBAS vector only; lane 3, ferritin (positive control); lane 4, glucose; lane 5, glycerol; lane 6, barley; lane 7, starch; and lane 8, molasses. Bacillus subtilis transformants containing pRBASFer-ori were cultured in a 100 mL PY medium by using a 500 mL baffled flask.

After culturing for 48 hours by adding various nitrogen sources (soy peptone, peptone, potassium nitrate, corn steep liquor, defatted soybean meal, soy protein isolate) to the basic medium at a concentration of 1% to 4%, the protein of the fermentation broth was analyzed by using SDS-PAGE and western blot. When soy peptone (2%) as a nitrogen source was used, the protein was secreted at the highest level (up to 20% of total proteins), compared to other nitrogen sources, and although the culture time was increased, the ferritin degradation by proteolytic enzymes did not occur in Bacillus subtilis LKS87 containing pRBASFer-ori(shown in FIG. 4(A)), in contrast to the case when ferritin was secreted from the Bacillus subtilis 168 (wild type) to which same pRBASFer-ori vector was introduced (data not shown). This may be caused because the B. subtilis LKS87 (nprR2, nprE18, aprA3, amyE) strain used in the fermentation lacked two protease genes and so the partial degradation of foreign proteins by the activation of different protases did not occur even during the culture time was extended to the resting phase in the growth of Bacillus subtilis. In addition, considering the raw material price of nitrogen sources for mass culture, using soy peptone at a high concentration realistically gives a significant impact of the unit price. Therefore, the concentration of soy peptone as a nitrogen source was fixed to 2% in the basic PY medium, and the carbon sources (glucose, glycerol, barley, starch and molasses) were each added at a concentration of 1% to 2% to measure the effect of carbon source on the ferritin secretion. As shown in FIG. 4(B), the results showed that the best secretion efficiency of the ferritin was found at 2% glucose (21% of total protein) and barley (21.5% of total protein) as a carbon source. Although the culture conditions established in batch culture may not be exactly the same as the conditions for mass production, based on the results obtained, mass culture was performed at an appropriate temperature)(30° C. in an optimized medium (PY medium+2% soy peptone and 2% barley) by using a 5 L jar-fermenter and a 50 L fermenter to measure the cell density and the amount of ferritin secretion depending on the culture time.

### 2.3. Mass Culture and Protein Analysis

The Bacillus subtilis transformant (containing pRBASFer-ori) cultured in an optimized medium by using a 5 L jar-fermenter under the conditions of 30° C., 200 rpm, and 1 vvm for 72 hours to measure the cell density and the amount of ferritin secretion.

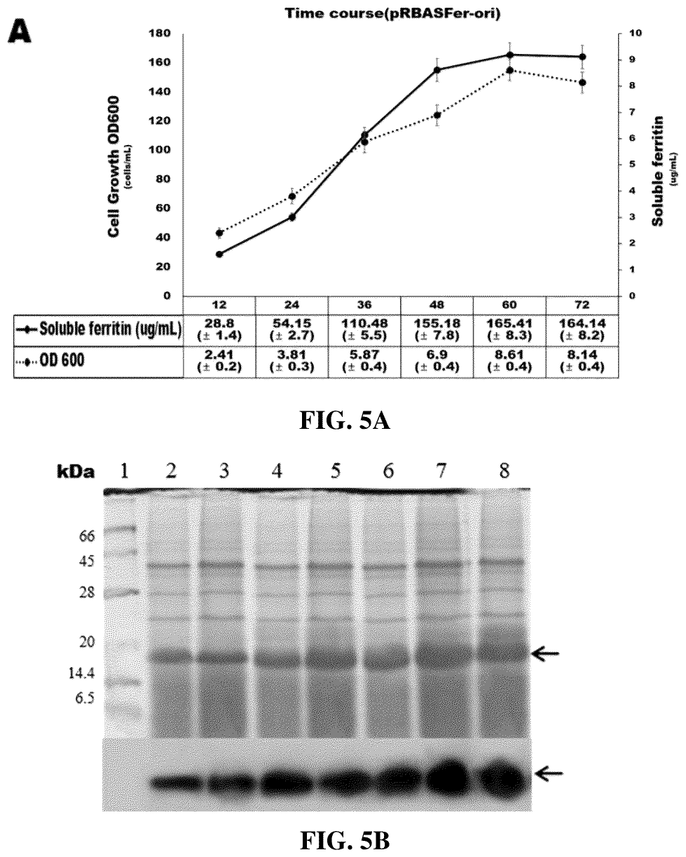

FIG. 5(A) and 5(B) shows the ferritin production of the Bacillus subtilis transformant containing pRBASFer-ori during culture times. FIG. 5(A) shows the secreted ferritin protein and the cell growth according to the time interval, and FIG. 5(B) shows the analytical results from SDS-PAGE and Western blotting.

In FIG. 5(B), lane 1, MW marker; lane 2, ferritin (positive control); lane 3, pRBASFer-ori (6 h); lane 4, pRBASFer-ori (12 h); lane 5, pRBASFer-ori (24 h); lane 6, pRBASFer-ori (36 h); lane 7, pRBASFer-ori (48 h); lane 8, pRBASFer-ori (60 h); and lane 9, pRBASFer-ori (72 h).

Soy peptone (2%) and barley (2%) were added to a PY medium, and culture at 30° C. for 72 hours. Samples were taken every 12 hours and the secreted ferritin protein content was analyzed.

As shown in FIG. 5(A), the cell density reached the highest maximum value (OD₆₀₀=8.61) when the fermentation time reached to 60 hours incubation. The amount of ferritin protein secretion started to increase from the 12th hours (28.8 μg/mL), and the secretion was highest as 165.4 μg/mL at the 60th hours incubation. Therefore, the ferritin production and secretion increased in proportion to the cell density, and degradation of foreign proteins by proteases was not found. Based on these results, mass culture was attempted in a 50 L fermenter for industrial use. As in the case of the 5 L jar-fermenter, the 50 L mass culture (working volume 30 L) was performed by inoculating B. subtilis LKS87 containing pRBASFer-ori to an optimized medium (PY+2% soy peptone+2% barley) at a concentration of 1% and culturing under the conditions of 30° C., 150 rpm, and 1 vvm for 72 hours to measure the cell density and the amount of ferritin secretion.

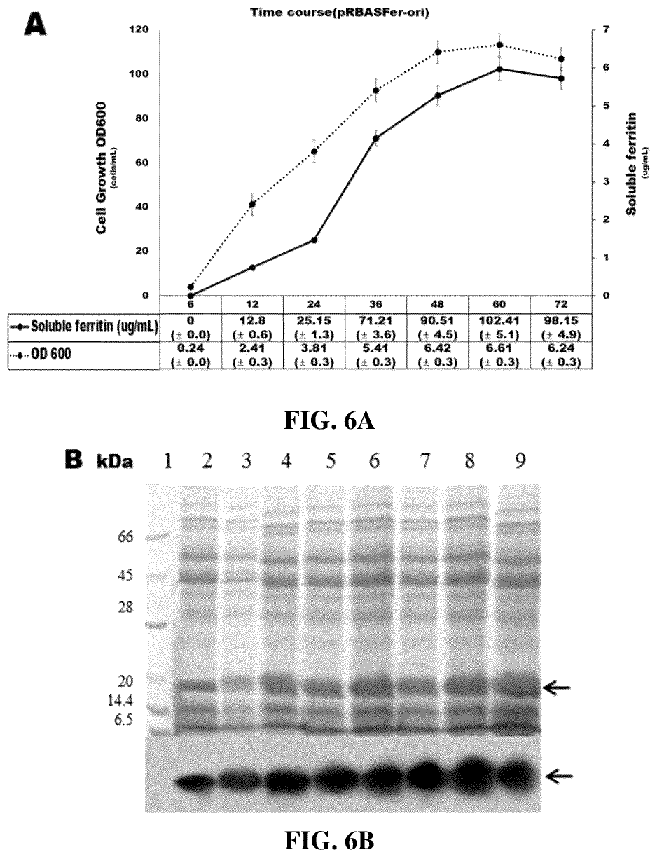

FIG. 6(A) and 6(B) shows the ferritin production of the Bacillus subtilis transformant containing pRBASFer-ori in a 50 L jar-fermenter, wherein FIG. 6(A) shows the secreted ferritin protein and the cell growth according to the time interval and FIG. 6(B) shows the analytical results from SDS-PAGE and Western blotting.

In FIG. 6(B), lane 1, MW marker; lane 2, ferritin (positive control); lane 3, pRBASFer-ori (6 h); lane 4, pRBASFer-ori (12 h); lane 5, pRBASFer-ori (24 h); lane 6, pRBASFer-ori (36 h); lane 7, pRBASFer-ori (48 h); lane 8, pRBASFer-ori (60 h); and lane 9, pRBASFer-ori (72 h).

Soy peptone (2%) and barley (2%) were added to a PY medium, and cultured at 30° C. for 72 hours. Samples were taken every 12 hours and the secreted ferritin protein content was analyzed.

As shown in FIG. 6(A), the cell density increased with the fermentation time and reached a maximum value (OD₆₀₀=6.61) when fermentation time reached 60 hours. The amount of ferritin secretion also increased from the 12th hour (12.8 μg/mL), and the secretion was highest as 102.4 μg/mL at the 60th fermentation hour. The amount of ferritin secretion was lower by about 38%, compared to the 5 L jar-fermenter (165.4 μg/mL) at the same time (60th hour), but the difference may be caused because of the decrease of the dissolved oxygen supply due to the difference in the stirring speed. However, with regard to the total amount obtained by the one-time fermentation, a sufficient amount of ferritin protein could be obtained in order to administrate each livestock with the appropriate dose. After the completion of the fermentation, the cells were removed by using a continuous centrifuge and the fermentation broth was recovered to prepare a liquid type feed additive to be fed to the feed or drinking water at a concentration of about 0.1% (6 μg/mL). The effect on the egg quality of laying hens was measured to investigate the biological activity of the ferritin protein (iron supplement). In addition, as in the case of the 5 L jar-fermenter, no ferritin degradation by proteases was found in the ferritin production performed by using the 50 L fermenter.

### 2.4. Comparison of Ferritin Activity with an Existing Recombinant Ferritin Protein Containing an Iron-inding Active Polypeptide Derived From Perinereis aibuhitensis

In addition, in order to compare the ferritin activity and the possibility of mass production between the ferritin containing the iron-binding active polypeptide derived from Perinereis aibuhitensis of KR Patent Registration No. 10-1253505 (registered on Apr. 5, 2013) (hereinafter referred to as Comparative Example 1) and the Periserrula leucophryna-derived ferritin according to the present invention, the inventors of the present invention performed mass culture of the transformant to which the transformation vector pHPS-Fer disclosed in Example 6 of the publication of the KR Patent Registration No. 10-1253505 (registered on Apr. 5, 2013) under the same mass culture conditions described in 2.3 above, and then analyzed the cell density and the amount of ferritin secretion.

After 1% inoculation to the medium (PY+2% soy peptone+2% barley) in a 50 L (working volume 30 L) jar-fermenter for mass culture, the culture was performed under the conditions of 30° C., 150 rpm and 1 vvm for 72 hours.

Table 1 below shows the amount of ferritin secretion and the cell density of the ferritin containing the iron-binding active polypeptide derived from Perinereis aibuhitensis of KR Patent Registration No. 10-1253505 (registered on Apr. 5, 2013).

TABLE 1

| | 6 h | 12 h | 24 h | 36 h | 48 h | 60 h | 72 h |
|---|---|---|---|---|---|---|---|
| Soluble | 0 | 6.7 | 12.14 | 50.76 | 65.27 | 70.82 | 67.49 |
| ferritin | (±0.0) | (±0.3) | (±0.7) | (±2.5) | (±3.2) | (±3.6) | (±3.5) |
| (ug/mL) | | | | | | | |
| OD 600 | 0.07 | 1.14 | 2.42 | 3.48 | 4.79 | 5.14 | 5.07 |
| | (±0.0) | (±0.3) | (±0.3) | (±0.3) | (±0.3) | (±0.3) | (±0.3) |

The results showed that in Comparative Example 1, as in the case of the ferritin protein according to the present invention, the amount of ferritin secretion started to increase from the 12th hour of the fermentation time and was highest at the 60th hour, and the amount of ferritin secretion and the cell density were 70.82 μg/mL and 5.14, respectively, at the 60th hour.

On the contrary, when the Bacillus subtilis transformant according to the present invention was used, the amount of ferritin secretion and the cell density were 102.41 μg/mL and 6.61, respectively, which were about 31% and about 28% higher than those of Comparative Example 1, indicating that tidal flat organisms of different species have different ferritin expression and secretion characteristics. In particular, the present invention showed better results because, in pursuit of more efficient expression and secretion of the ferritin gene obtained from Periserrula leucophryna, the expression and secretion were optimized by introducing an additional replication origin through an expression secretion vector comprising the key regulatory factors, such as promoter, Shine-dalgarno (SD), operator, signal sequence and replication origin, in other words, it is judged that the optimization of ferritin expression and secretion may be achieved by increasing the copy number of palsmid in Bacillus subtilis.

### 2.5. Biological Activity of Ferritin in Laying Hens

Iron feeds that are generally used are mineral-added feed containing calcium phosphate and ferrous sulfate, but reports have shown that organic iron has a higher bioavailability than mineral iron in view of the digestion and absorption of iron. Therefore, feed additive containing a ferritin liquid type with organic iron (6 μg/mL) was fed on voluntary intake for 6 weeks as an amount corresponding to 0.1% of drinking water by using an automatic water supply facility of a laying hen (ISA Brown, 80 weeks old, 300 laying hens) farm. After that, 60 eggs were taken every week to analyze the eggshell color, egg yolk weight, Haugh unit (freshness), and iron content in the eggshell and egg yolk. The amounts of the drinking water and ferritin during the breeding period were measured as follows: The average amount of daily drinking water intake by a single laying hen was approximately 400 mL, that is, about 5,040 L of drinking water to the 300 laying hens for 6 weeks. Therefore, the amount of ferritin protein needed (6 μg/mL) was about 5.04 L (30.3 mg ferritin).

Table 2 below shows the biological activity of the ferritin in laying hens

TABLE 2

| | | | | | Height of | Height of | Egg white | Yolk sac | | | |
|---|---|---|---|---|---|---|---|---|---|---|---|
| | Eggshell color | | | W | egg white | yolk sac | diameter | diameter | Albumin | Yolk | Haugh |
| Sample | L* | a* | b* | g | mm | mm | Mm | mm | index | index | unit |
| Control | 73.23 | 9.55 | 25.72 | 57.82 | 4.50 | 15.63 | 69.35 | 38.98 | 0.06 | 0.40 | 64.44 |
| | (±1.1) | (±1.4) | (±1.0) | (±1.8) | (±0.6) | (±0.6) | (±0.6) | (±0.6) | (±0.01) | (±0.02) | (±2.73) |
| 6 weeks | 62.53 | 18.39 | 32.00 | 66.46 | 5.56 | 18.57 | 75.52 | 40.6 | 0.07 | 0.46 | 70.52 |
| | (±1.1) | (±1.5) | (±1.1) | (±1.6) | (±0.3) | (±0.5) | (±0.6) | (±0.6) | (±0.01) | (±0.01) | (±1. [symbol] 2) |

Haugh unit: 100 × log (h · 1.7m [symbol] 7.6)
W: Egg weight [symbol]
indicates data missing or illegible when filed

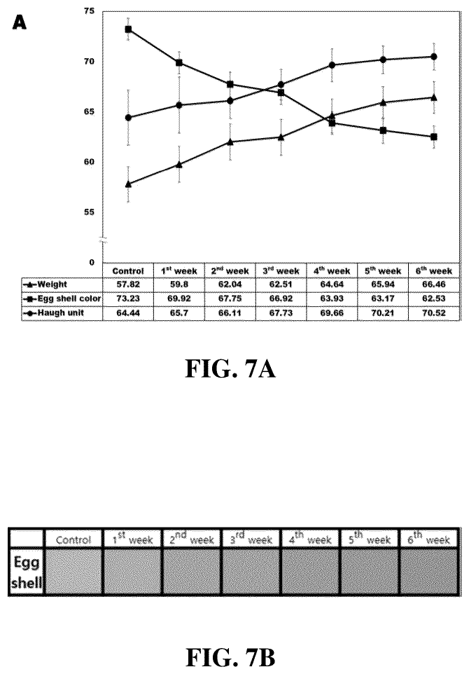

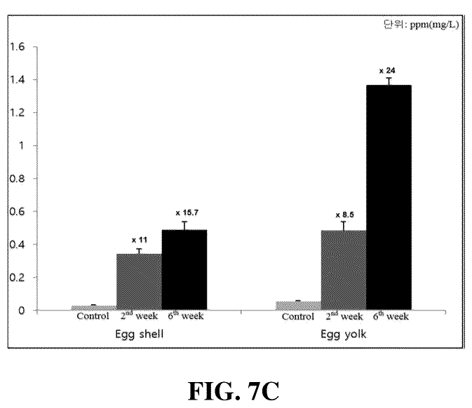

FIG. 7(A), 7(B) and 7C) shows the effect of mass-produced ferritin protein on egg quality (egg color, egg weight, and Haugh unit (freshness)), wherein FIG. 7(A) is a plot showing the effect of the ferritin protein on the quality of eggs (eggshell color, egg weight, Haugh unit (freshness)) and the change for 6 weeks; FIG. 7(B) shows the 6-week change of the color of the eggshell treated with the ferritin protein; and FIG. 7(C) is a plot showing the iron content in the eggshell and egg yolk measured by using Inductively Coupled Plasma (ICP) and the change over time.

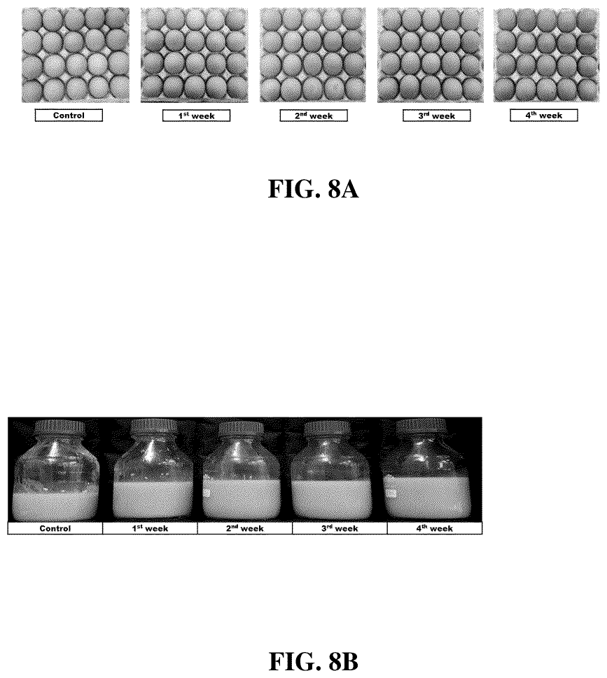

FIG. 8(A) and 8(B) shows the color change of (A) the eggshell and (B) the egg yolk from Week 1 to Week 4. The eggshell color change from bright orange to brown was clearly observed over time, and the color of the egg yolk was slightly changed to darker orange.

As shown in FIG. 7(A), the measurement of the weight of the eggs following the administration of ferritin-containing drinking water for 6 weeks showed that the egg weight was increased each week, and the egg weight after 6 weeks (66.46 g) was about 15% higher than that of the control group (57.82 g). The analysis of the eggshell color based on CIE (L*a*b*) showed that the eggshell color of the control group before the ferritin administration was light brown (73.23, 9.55, 25.72), and it was changed to dark brown (65.23, 18.39, 32) after 6 weeks, becoming about 17% darker (Table 2 and FIG. 7(B)). According to Table 2, the Haugh unit (HU, freshness) was 8% higher in the eggs administered with the ferritin for 6 weeks (70.52) than the control group (64.44). In addition, as shown in FIG. 7(C), the iron content in the eggshell and egg yolk, which was analyzed by ICP (inductively coupled plasma), was about 15.7 times and 24 times higher, respectively, in the eggs administered with the ferritin for 6 weeks than the control group. Although there were slight differences depending on the environmental conditions of the breeding farm, the results showed that the addition of the liquid type ferritin formulation generally have a significant effect on the quality of the eggs in terms of egg weight, eggshell and egg yolk color, iron content and freshness.

### 2.6 Biological Activity of Ferritin in Broilers

The biological activity of ferritin was confirmed with broilers (8 to 10 weeks of age). Table 3 below shows the biological activity when ferritin is supplied in a liquid form.

TABLE 3

| | | No. | | Culturing | No. | Rate of | | | | | | |
|---|---|---|---|---|---|---|---|---|---|---|---|---|
| | Input | Input | Output | Period | Output | Survival | TBW | ABW | ADG | TFI | | |
| Sample | Date | animal | Date | (Days) | animal | (%) | (kg) | (kg) | (g) | (kg) | FE | PI |
| Control | 2019 | 9,000 | 2019 | 34 | 8,784 | 97.6% | 12,912 | 1.47 | 43.23 | 22,467 | 1.74 | 242.5 |
| | Nov. 27 | | Dec. 31 | | | | | | | | | |
| Treated | 2019 | 9,000 | 2019 | 34 | 8,874 | 98.6% | 14,110 | 1.59 | 46.76 | 21,871 | 1.55 | 297.5 |
| group | Nov. 27 | | Dec. 31 | | | | | | | | | |

A ferritin composition containing 5 mg of ferritin per 1 L of liquid feed was supplied to drinking water to a final concentration of 0.1%.

Table 4 below shows the biological activity when ferritin is supplied in a powder form.

TABLE 4

| | | No. | | Culturing | No. | Rate of | | | | | | |
|---|---|---|---|---|---|---|---|---|---|---|---|---|
| | Input | Input | Output | Period | Output | Survival | TBW | ABW | ADG | TFI | | |
| Sample | Date | animal | Date | (Days) | animal | (%) | (kg) | (kg) | (g) | (kg) | FE | PI |
| Control | 2019 | 10,200 | 2019 | 30 | 9,690 | 95.0% | 14,922 | 1.54 | 51.3 | 25,008 | 1.68 | 290.3 |
| group | Feb. 23 | | Mar. 25 | | | | | | | | | |
| Treated | 2019 | 9,900 | 2019 | 30 | 9,700 | 98.0% | 15,617 | 1.61 | 53.7 | 23,998 | 1.54 | 341.5 |
| group | Feb. 23 | | Mar. 25 | | | | | | | | | |

A ferritin composition containing 5 mg of ferritin per 1 kg of wheat sorts was supplied to a final concentration of 0.1% for the main feed on feeding.

Here, ADG is the average daily gain, ABW is the average body weight, culturing period is the feeding period, FE is the feed efficiency, TFI is the total feed intake, TBW is the total weight and PI is the production index. Average daily gain (ADG), feed efficiency (FE), and production index (PI) were calculated as described below.

ADG (Average daily gain): ABW (Average body weight)/Culturing Period

FE (Feed efficiency): TFI (Total feed intake)/TBW (Total body weight)

PI (Production index): (ABW×Rate of Survival)/(FE×Culturing Period)×100

The results showed that the survival rate and the production index were higher in the group to which the ferritin composition was supplied than in the non-treatment group. The PI was higher when the ferritin composition was supplied in the form of powder (22.68%) than in the form of liquid (17.63%).

### 2.7. Biological Activity of Ferritin in Pig Breeding

The biological activity in piglets was confirmed in weanling pigs of three-way crossbreeds (Landrace×Yorkshire×Duroc). The piglets were bred for 6 weeks, and corn-soybean meal was supplied as a basal diet, and water was fed on voluntary intake.

Table 5 below shows the effect of mass-produced ferritin protein on the growth capacity of the weanling pigs.

TABLE 5

| | | | | | | P-Value |
|---|---|---|---|---|---|---|
| | | | | | | NC vs |
| Items | NC | T1 | T2 | T3 | SE² | Ferritin |
| Body | | | | | | |
| weight (kg) | | | | | | |
| Initial | 7.74 | 7.71 | 7.71 | 7.72 | 0.04 | 0.486 |
| d 14 | 12.64^{c} | 12.85^{bc} | 13.33^{ab} | 13.58^{a} | 0.19 | 0.014 |
| d 42 | 28.08^{b} | 28.7^{b} | 29.66^{a} | 30.52^{a} | 0.28 | 0.0002 |
| Phase 1 | | | | | | |
| (0-14 d) | | | | | | |
| ADG, g | 350^{c} | 367^{bc} | 402^{ab} | 419^{a} | 15 | 0.015 |
| ADFI, g | 503 | 497 | 496 | 504 | 7 | 0.831 |
| G/F | 0.695^{b} | 0.739^{ab} | 0.811^{a} | 0.832^{a} | 0.035 | 0.027 |
| Phase 2 | | | | | | |
| (15-42 d) | | | | | | |
| ADG, g | 551^{c} | 566^{bc} | 583^{ab} | 605^{a} | 7 | 0.001 |
| ADFI, g | 831 | 831 | 824 | 832 | 10 | 0.858 |
| G/F | 0.664^{c} | 0.681^{bc} | 0.709^{ab} | 0.727^{a} | 0.014 | 0.019 |
| Overall | | | | | | |
| (0-42 d) | | | | | | |
| ADG, g | 48.4^{b} | 500^{b} | 523^{a} | 543^{a} | 7 | 0.0002 |
| ADFI, g | 722 | 720 | 715 | 723 | 7 | 0.755 |
| G/F | 0.671^{c} | 0.694^{bc} | 0.733^{ab} | 0.751^{a} | 0.013 | 0.002 |
| | | | | (0.0 [symbol] ) ↑ | | |

²Standard error
^{a, b, c}Means in the same row with different superscripts differ (P < 0.05) [symbol]
indicates data missing or illegible when filed

The body weight was measured at the initial state and Week 2 and Week 6, wherein ADG is the average daily gain, ADFI is the average daily feed intake, and Growth/Feed (G/F) ratio is the feed efficiency.

Here, NC (Normal Control) is a basal diet supply group; T1 is NC+0.1% ferritin low concentration <5.0 mg/kg; T2 is NC+0.1% ferritin 10 mg/kg; and T3 is NC+0.1% ferritin 15 mg/kg.

The results confirmed that the average daily weight gain and the growth rate were increased, as the ferritin protein supply content and the concentration were increased.

Table 6 below shows the effect of mass-produced ferritin protein on blood characteristics of weanling pigs.

TABLE 6

| | | | | | | P-Value |
|---|---|---|---|---|---|---|
| | | | | | | NC vs |
| Items | NC | T1 | T2 | T3 | SE² | Ferritin |
| Week 2 | | | | | | |
| Fe, ug/dL | 98^{b} | 120^{a} | 124^{a} | 139^{a} | 7 | 0.003 |
| WBC, 10³/ [symbol] | 7.49^{b} | 12.69^{a} | 11.23^{ab} | 13.26^{a} | 1.23 | 0.005 |
| RBC, 106/ [symbol] | 2.65 | 2.41 | 2.40 | 2.83 | 0.15 | 0.963 |
| Lymphocyte, % | 59.2 | 48.1 | 46.0 | 54.5 | 7.7 | 0.302 |
| Week 6 | | | | | | |
| Fe, ug/dL | 118^{b} | 137^{ab} | 148^{ab} | 151^{a} | 10 | 0.033 |
| WBC, 10³/ [symbol] | 14.83 | 10.94 | 13.34 | 11.96 | 1.63 | 0.170 |
| RBC, 10⁶/ [symbol] | 3.77 | 3.43 | 5.61 | 4.55 | 0.77 | 0.407 |
| Lymphocyte, % | 44.9^{a} | 35.2^{b} | 34.3^{b} | 40.73^{ab} | 2.6 | 0.021 |
| Hemoglobin, g/dL | 5.8 | 5.1 | 6.0 | 6.0 | 0.81 | 0.937 |
| TIBC, ug/dL | 558 | 606 | 833 | 676 | 38 | 0.090 |

²Standard error;
TIBC: Total Iron Binding Capacity
^{a, b, c}Means in the same row with different superscripts differ (P < 0.05)

An automatic blood analyzer (ADVID120, Bayer, USA) was used to measure WBC, RBC, lymphocyte and hemoglobin. An automatic serum biochemical analyzer (Hitachi 747, Japan) was used to measure the serum Fe and TIBC (total iron binding capacity).

Here, NC (Normal Control) is a basal diet supply group; T1 is NC+0.1% ferritin low concentration <5.0 mg/kg; T2 is NC+0.1% ferritin 10 mg/kg; and T3 is NC+0.1% ferritin 15 mg/kg.

The results showed that the iron content was increased depending on the increase of the amount and concentration of the supplied ferritin protein. In Week 6 of the feeding, the hemoglobin level was highest as 6 g/dL in the T2 group and the T3 group, and the TIBC was increased with the increase of the concentration. In Week 2, the RBC and WBC levels were high in the T3 group wherein the ferritin protein concentration was high. In Week 6, however, the WBC level was highest in the basal diet supply group, and the RBC was highest in the T2 group, and so a distinctive trend of the numerical values was not found with the increase of the concentration. The lymphocyte level was higher in the basal diet supply group than those in both Week 2 and Week 6 of the feeding, however, indicating that the difference of lymphocyte values may not reflect healthy status.

The application of the ferritin protein to weanling pigs, laying hens and broilers for confirming the effect of ferritin protein as a feed additive showed that an increase of the survival rate and production index was confirmed in broilers. In the case of weanling pigs, an increase of the ADG and the G/F ratio was confirmed. In the laying hens, distinctive browning of the eggshell color and an increase of the egg weight and freshness were confirmed. In particular, the effect was clearly shown in the laying hens fed with the ferritin protein, as the iron content in the eggshell and egg yolk was increased by 15.7 times and 24 times, respectively.

In the present invention, in order to industrially apply the iron protein ferritin derived from Periserrula leucophryna, mass culture was performed by optimizing the secretion efficiency in Bacillus subtilis, and a system was established to utilize the fermentation broth as a feed additive after removing the cells. Replication origins were added in a tandem arrangement to the previously constructed pRBASFer secretion vector to increase the copy number, and the constructed pRBASFer-ori vector was introduced to B. subtilis LKS87. After that, the ferritin protein production was optimized, and so the production yield was increased by about 1.4 times.

The prepared ferritin protein was applied to laying hens, broilers, and weanling pigs to verify the biological activity. The results confirmed an increase of the survival rate and production index in broilers, and an increase of ADG and G/F ratio in weanling pigs.

To verify the biological activity of ferritin in laying hens, the ferritin was formulated in a liquid type and then added to drinking water. The key indicators of the egg quality from laying hens, which are eggshell color, freshness, and iron content in eggshell and egg yolk, were measured, and the results showed that all the numerical values were increased. In particular, the iron content measurement showed that the iron content was increased 11 to 15.7 times in the eggshell and 8.5 to 24 times in the egg yolk, compared to the control group. Therefore, the ferritin-containing fermentation broth, having a sufficiently high biological activity as a feed additive, is expected to be competitive in the field of feed and be useful in the fields of food industry and pharmaceutical material industry for the prevention and treatment of iron deficiency.

As described above, the present invention has been mainly described with reference to the accompanying drawings, but those of ordinary skill in the art to which the present invention pertains within the range not departing from the technical principles and the scope described in the claims of the present invention may practice various modifications or variations of the present invention. Therefore, the scope of the present invention should be construed by the appended claims that are described to include examples of the many modifications.

What is claimed is:

1. A mass production method of Periserrula leucophryna-derived ferritin protein using a recombinant vector for mass production of ferritin protein, the recombinant vector comprising a gene encoding Periserrula leucophryna-derived ferritin protein represented by an amino acid sequence of SEQ ID NO: 1, the method comprising:
1) cloning, into a T vector, a DNA fragment of the replication origin obtained by performing PCR by using a pRBASFer vector, which is a vector comprising a ferritin gene of Periserrula leucophryna, as a template;
2) obtaining a DNA fragment of the replication origin from the T vector into which the DNA fragment is cloned;
3) preparing new recombinant secretion vector (pRBASFer-ori) in which the replication origin is introduced in a tandem array by digesting the existing pRBASFer vector with a restriction enzyme and filling with a Klenow enzyme, and again digesting with another restriction enzyme and ligating the DNA fragment obtained in 2) above; and
4. culturing, in a medium, a Bacillus subtilis transformant into which the recombinant vector is introduced,
wherein, in 1) above, PCR is performed with the existing pRBASFer vector as a template and with a forward primer of SEQ ID NO: 5 and a reverse primer of SEQ ID NO: 6, which have an introduced restriction enzyme site and which are specific to a replication origin in the pRBASFer vector.
wherein, in 4) above, the Bacillus subtilis transformant to which the recombinant vector is introduced is cultured at a culture temperature of 28 to 35° C. at a stirring speed of 150 to 250 rpm, at an air injection rate of 0.5 to 2 vvm for 36 to 96 hours incubation by adding, into the medium, soy peptone 1% to 4% (w/v) as a nitrogen source and barley 1% to 3% (w/v) as a carbon source.

2. A composition to be added to main feed, comprising ferritin protein produced by the mass production method of Periserrula leucophryna-derived ferritin protein according to claim 1,
wherein the composition to be added to main feed is a feed additive for laying hens, broilers, and weanling pigs; and
wherein the ferritin protein is added, based on 100 parts by weight of main feed and drinking water, in an amount of 0.01 to 5 parts by weight.

3. The composition to be added to main feed comprising ferritin protein produced by the mass production method of Periserrula leucophryna-derived ferritin protein according to claim 1,
wherein the composition to be added to main feed is one of a feed additive for laying hens, a feed additive for broilers, and a feed additive for weanling pigs,
wherein the effect of composition to be added to main feed on laying hens is verified by a biological activity that eggshell color, freshness, and iron content in eggshell and egg yolk of eggs laid from laying hens are all increased,
wherein the effect of composition to be added to main feed on broilers is verified by a biological activity that survival rate, feed efficiency and production index of broilers are increased; and
wherein the effect of composition to be added to main feed on weanling pigs is verified by a biological activity that average daily gain and growth/feed (G/F) ratio of weanling pigs are increased.


SEQUENCE LISTING (6 sequences)

SEQ ID NO: 1 - protein, 174 aa, Periserrula leucophryna
  REGION 1..174: note = Ferritin of Periserrula leucophryna
MATSRQTMPRQNYHEECEAGINKQINLELYASYVYQSMAWYFNRDDVALPGFHHFFKKASEEEREHAEKFMKYQNMRGGRIVLQDIKKPERDEWGTGLEAMQAAHALEKHVNQSLLDLHKLADGHDDGQLTDFLEGEYLKEQVEAIKEISDHITQLKRVGPGLGEYMYDKELKS

SEQ ID NO: 2 - DNA, 1111 bp, Periserrula leucophryna
  misc_feature 1..1111: note = Ferritin of Periserrula leucophryna
  source 1..1111: mol_type = unassigned DNA
aaaactttgatatcgactgtatcttgggacgtcagtgtgcgtacggatcggcggtatctcttcttcaaaacatcctaaccatttctattcaacgtcgttaagtcgagttcaataggtgcgaactaaagatggccacatccagacaaaccatgccccgccagaactaccatgaggagtgcgaagctggaatcaacaaacagatcaatctcgaactctacgccagctatgtttaccaatctatggcatggtacttcaacagggatgatgttgccctcccaggcttccatcatttcttcaagaaggcttctgaggaagaacgcgaacatgctgagaagttcatgaagtaccagaacatgaggggtggtcgtatcgttctgcaggacatcaagaagccggagagggatgagtggggaactggattggaggccatgcaagcggcccatgcactggagaagcatgtcaaccagtccctgcttgatcttcacaagttggctgatggccacgatgacggccagctgactgacttcctggagggcgagtacctcaaggaacaagtagaggcaatcaaggagatcagcgaccacatcacccagctgaaacgtgtcggtcccggcctgggagagtacatgtacgacaaggaactcaagagctagatgacctacctataaggtcaagagcctcaggccgtcaccgagacgccaagcatagacccacaatactctcgctgcagtcttatctccttagcctacccctgtatgaatcaacatctttgtttgctatagaatagtcatcagtaacaacattctcttaatatctatgatttctgcttagtgtggttcaagtttaatacttcttaaaagttacacatggcacggcatgtgaagataatggcatctgtagtttcacatattgcttctgaactgtaccatagtcaggaaacttaatataacattcctaatgtcaaatccaggtacatttatcaaatttgtttcaatcttgtgtaacatttgaaactgccctggtggtagaagtttgaaaagaaaggccttggtattgttcagttgtgttgaatagatgcctaaaaaaaaaaaaaaaaaaaaaaaaaaaaaaa

SEQ ID NO: 3 - DNA, 525 bp, unidentified
  misc_feature 1..525: note = coding nucleotide, Ferritin of Periserrula leucophryna
  source 1..525: mol_type = unassigned DNA
atggccacatccagacaaaccatgccccgccagaactaccatgaggagtgcgaagctggaatcaacaaacagatcaatctcgaactctacgccagctatgtttaccaatctatggcatggtacttcaacagggatgatgttgccctcccaggcttccatcatttcttcaagaaggcttctgaggaagaacgcgaacatgctgagaagttcatgaagtaccagaacatgaggggtggtcgtatcgttctgcaggacatcaagaagccggagagggatgagtggggaactggattggaggccatgcaagcggcccatgcactggagaagcatgtcaaccagtccctgcttgatcttcacaagttggctgatggccacgatgacggccagctgactgacttcctggagggcgagtacctcaaggaacaagtagaggcaatcaaggagatcagcgaccacatcacccagctgaaacgtgtcggtcccggcctgggagagtacatgtacgacaaggaactcaagagctag

SEQ ID NO: 4 - DNA, 28 bp, unidentified
  misc_feature 1..28: note = iron responsive element (IRE)
  source 1..28: mol_type = unassigned DNA
atcttgggacgtcagtgtgcgtacggat

SEQ ID NO: 5 - DNA, 30 bp, synthetic construct
  misc_feature 1..30: note = forward primer
  source 1..30: mol_type = other DNA
gggacatgttctttcctgcgttatcccctg

SEQ ID NO: 6 - DNA, 30 bp, synthetic construct
  misc_feature 1..30: note = reverse primer
  source 1..30: mol_type = other DNA
cccgatatcctatttagaatattgtttagt